(19) United States
(12) Patent Application Publication
(10) Pub. No.: US 2013/0253444 A1
(43) Pub. Date: Sep. 26, 2013

(54) Medical Needle Safety Device

(71) Applicant: Barry Peter Liversidge, Colchester Essex (GB)

(72) Inventor: Barry Peter Liversidge, Colchester Essex (GB)

(21) Appl. No.: 13/990,973

(22) PCT Filed: Dec. 2, 2011
(86) PCT No.: PCT/GB2011/052384

§ 371 Date: May 31, 2013

(30) Foreign Application Priority Data
Dec. 3, 2010 (GB) 1020542.5

(51) Int. Cl. A61M 5/32

(52) U.S. Cl. CPC A61M 5/3202
USPC 604/263

(57) ABSTRACT

A safety device 20 for shielding a medical needle 25 having a sharp tip includes a needle mount 28 which is adapted directly or indirectly to support the needle 25. A needle shielding sleeve 33 is arranged coaxially with the mount 28 for sliding movement between an initial shielding position surrounding the needle 25 and a non-shielding position at which at least the tip of the needle is exposed beyond the sleeve 33. Resilient fingers 35 urge the sleeve 33 to its needle shielding position. There is a control member 39 for the fingers 35 and which is slidably housed within the coaxial arrangement of the sleeve 33 and the mount 28. The resilient fingers 35 are essentially undeformed when the control member 39 is in its storage position (FIG. 3A) but are partially deformed when the control member is moved to a set position (FIG. 5A). A needle cover 42 is fitted on to the needle 25 to confer protection thereto but is removable from the needle prior to use of the device and is arranged so that removing the needle cover effects movement of the control member to its set position.

This invention relates to a safety device for use with a medical needle having a sharp tip, to confer passive protection to that needle. The invention extends to a safety needle assembly including such a device and to an injection device incorporating such a safety needle assembly.

This invention comprises an improvement to and modification of the safety needle devices described and claimed in my earlier International Patent Publication No. WO 2011/092518, claiming the priority of 1 Feb. 2010 and hereinafter referred to as “said application”. The entire description and drawings of said application are incorporated herein by this reference thereto.

In said application, there is described a medical needle safety device having a needle mount, a needle shielding sleeve movable from an initial shielding position to a non-shielding position and a radially deformable resilient finger provided on one of the sleeve and mount and having an initial substantially undeformed condition. There is a control member having a set position at which sliding movement of the sleeve from its initial position enables engagement of the finger with the control member to deform and lift the finger outwardly following which continued movement of the sleeve towards its non-shielding position increases the outward deformation of the finger. On subsequent return of the sleeve to its shielding position, the control member is moved from its set position and the finger returns to its substantially undeformed condition; thereafter the finger blocks movement of the sleeve towards its non-shielding position.

This invention stems from further development of the safety device of said application and in particular (but not exclusively) for a pre-filled syringe having a staked-in needle protected by a needle cover. The safety device of this preferred improvement has the particular aim of preventing operation of the device until the needle cover has been removed from the needle, in readiness for performing an injection.

According to a first aspect of this invention there is provided a safety device for shielding a medical needle having a sharp tip, which device comprises:
a needle mount for directly or indirectly supporting a medical needle;
a needle shielding sleeve for surrounding the needle and arranged coaxially with the mount so that a force applied to the sleeve slides the sleeve from an initial needle shielding position to a non-shielding position whereat at least the tip of the needle is exposed beyond the sleeve;
an abutment surface and a sliding surface provided on one of the sleeve and mount;
a radially deformable resilient finger provided on the other of the sleeve and mount, said finger being configured for radial alignment with the abutment surface or the sliding surface, a part of the finger being in radial alignment with the abutment surface when the finger is not radially deformed, thereby blocking movement of the sleeve from in its initial needle shielding position;
a control member slidably housed within the coaxial arrangement of the sleeve and mount and having a set position with respect thereto at which the control member engages with the finger to cause radial deformation thereof so allowing movement of the sleeve from its initial position, said deformation also causing a part of the finger to move into radial alignment with the sliding surface, and movement of the sleeve towards its non-shielding position causes increased deformation of the finger thereby generating a restorative force between the finger and the sliding surface that urges the sleeve back to its initial needle shielding position, said movement of the sleeve from the initial position causes the control member to be displaced from the set position and thereafter allows the finger to return into radial alignment with the abutment surface on return of the sleeve to its initial position, thereby blocking subsequent movement of the sleeve; and
displacement means arranged to effect movement of the control member from a starting position before use of the device to the set position.

Though the invention may be used with various designs of syringe and needle combinations, including needles having a mount or hub adapted for attachment to a syringe, the invention finds particular application with a pre-filled syringe having a pre-fitted (or staked-in) needle and fitted with a needle cover both to seal the sharp tip of the needle and also to confer sterility to the needle itself. In all preferred embodiments of this invention described hereinafter with reference to the drawings, the displacement means comprises the needle cover as is conventionally fitted to the needle of a pre-filled syringe having a staked-in needle as just described.

It will be appreciated that in this first aspect of this invention, the or each finger is initially in an undeformed condition (where the finger is not stressed), so the device, whether fitted to a syringe or not, may be stored indefinitely without the or each finger taking a set and so unable to return to its initial as-moulded and undeformed condition. After manufacture of the device, the control member is in a position where the or each finger remains undeformed, in radial alignment with the abutment surface of the sleeve. In this condition, said part of the or each finger blocks substantial movement of the sleeve away from its initial position until the device has been prepared for use by actuating the displacement means. With the preferred embodiments, the preparation comprises pulling a needle cover away from the device. This draws the control member to its set position whereat the or each finger is deformed radially outwardly away from its sleeve blocking position. Thereafter, the sleeve may be slid rearwardly with respect to the syringe and use of the device may proceed as has been described in said application.

The part of the or each finger which is in radial alignment with the abutment surface when the finger is undeformed may be the same part of the finger which moves into radial alignment with the sliding surface. Alternatively, the or each finger may have distinct parts, one for radial alignment with the abutment surface and another for engaging the sliding surface.

According to a second but closely related aspect of this invention, there is provided a safety device for shielding a medical needle having a sharp tip, which device comprises:
a needle mount supporting a medical needle or adapted directly or indirectly to support a medical needle;
a needle shielding sleeve for surrounding a supported needle and arranged coaxially with the mount for sliding movement between an initial needle shielding position and a non-shielding position whereat at least the tip of a supported needle is exposed beyond the sleeve;
resilient means urging the sleeve to its needle shielding position;
a control member for the resilient means, the control member being slidably housed within the coaxial arrangement of sleeve and mount and having a set position axially spaced from a storage position, the resilient means being in a substantially undeformed condition when the control member is in its storage position but being partially deformed when the control member in its set position; and
a needle cover fitted on to the needle to confer protection thereto but removable from the needle prior to use of the device and arranged so that removing the needle cover effects movement of the control member to its set position.

This second aspect of the invention is specifically adapted for use with a pre-filled syringe, where the needle of the syringe is fitted with a needle cover which both serves to give protection to the needle before use and also as a stopper for the sharp tip of the needle, to prevent leakage out of the needle of the drug pre-filled into the syringe. As an alternative, the invention may be used with a syringe intended for use with a needle having a hub which is attachable to the syringe when the syringe is to be used for performing a medical procedure.

Preferably, the needle cover is interconnected with but is releasable from the control member such that on removing the needle cover from its needle protecting position, the control member is moved to its set position whereafter continued movement of the needle cover away from the device releases the needle cover while leaving the control member in its set position.

At least the part of the needle cover interconnected with the control member, or the whole of the needle cover, may be resiliently deformable for passing through an opening in the control member. Then, when the control member reaches its set position, continued pulling of the needle cover deforms the rearward end of the cover sufficiently to allow the cover to pass through the opening and be freed from the device. The rearward end of the cover may be profiled so as to form a seal to the nose of a syringe or needle hub, in a manner known in the art.

The set position of the control member corresponds to the set position of the control member in said application. Thus, other features of the invention of said application are equally applicable to this invention and reference should be made to said application for a description thereof.

This invention extends to the combination of a syringe having a supported needle and a safety device of this invention as described above. The needle may be directly or indirectly supported by the syringe; in the case of the former, the needle may be staked-in so as to be permanently mounted to the nose of the syringe but in the case of the latter, the needle may be furnished with a needle hub which is releasably securable to the nose of a syringe for example by a Luer taper connector, a Luer lock connection or by co-operating screw threads.

Further, this invention extends to a medical injector including a source of liquid drug such as a vial or a syringe body, means to drive a plunger associated with the source of liquid drug and a needle projecting from the injector for performing an injection, in combination with a safety device of this invention as described above.

By way of example only, several specific embodiments of safety device of this invention will now be described in detail, reference being made to the accompanying drawings in which:

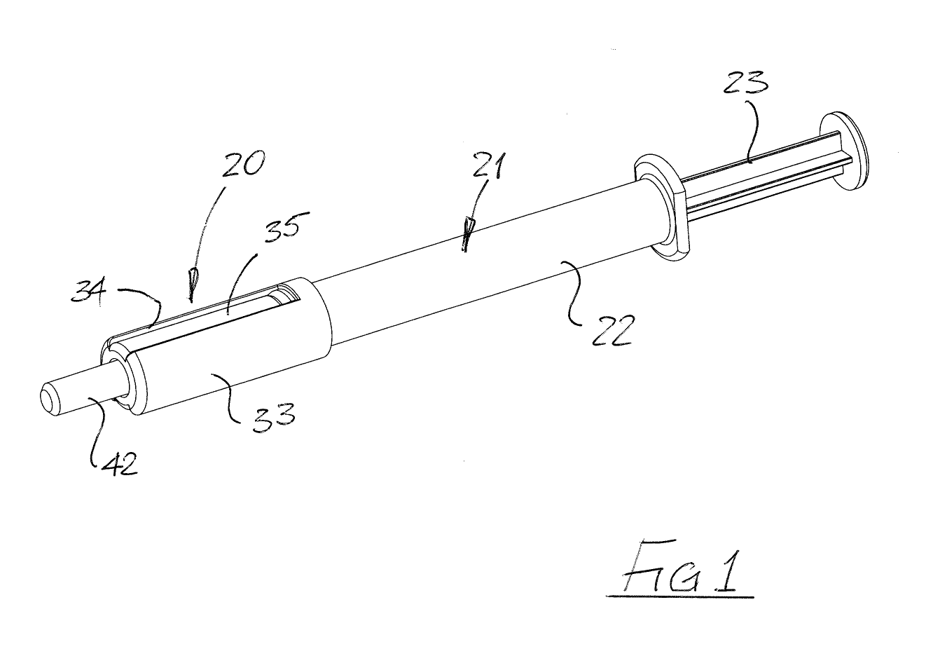

FIG. 1 is an isometric view of the first embodiment, fully assembled and ready for use;

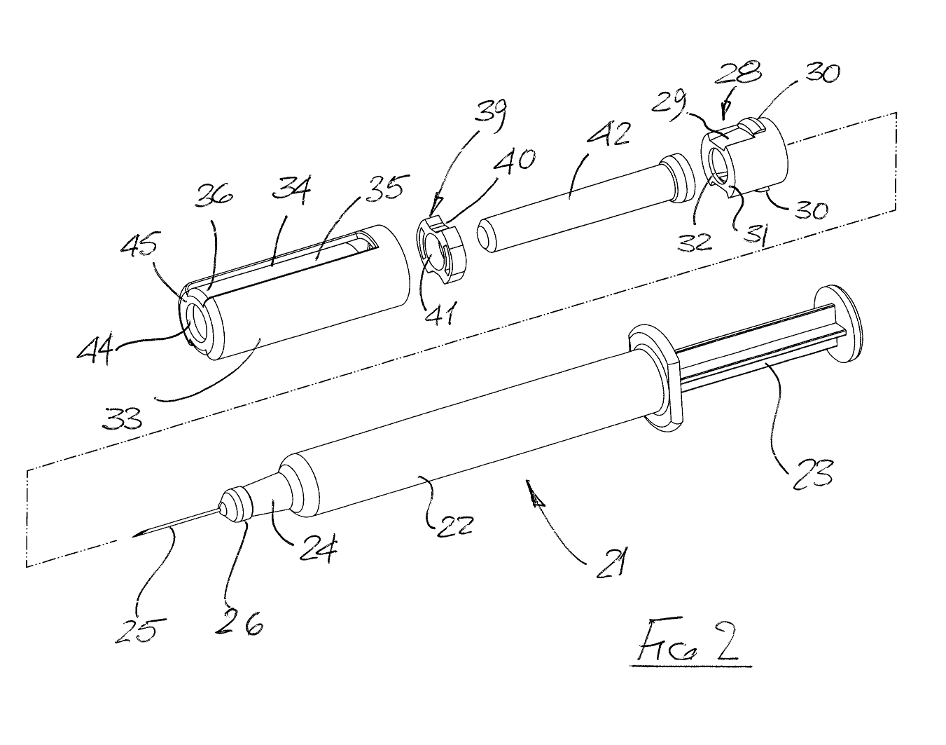

FIG. 2 is an exploded view of the first embodiment;

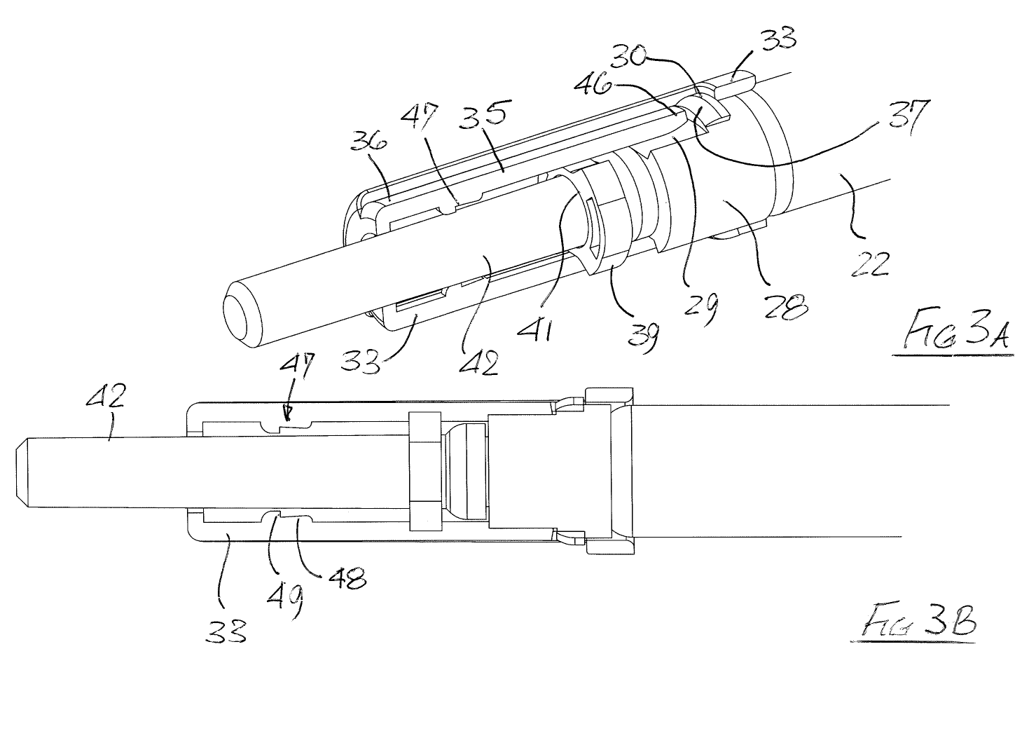

FIGS. 3A and 3B are respectively a cut-away isometric view and an axial section through the first embodiment, ready for use;

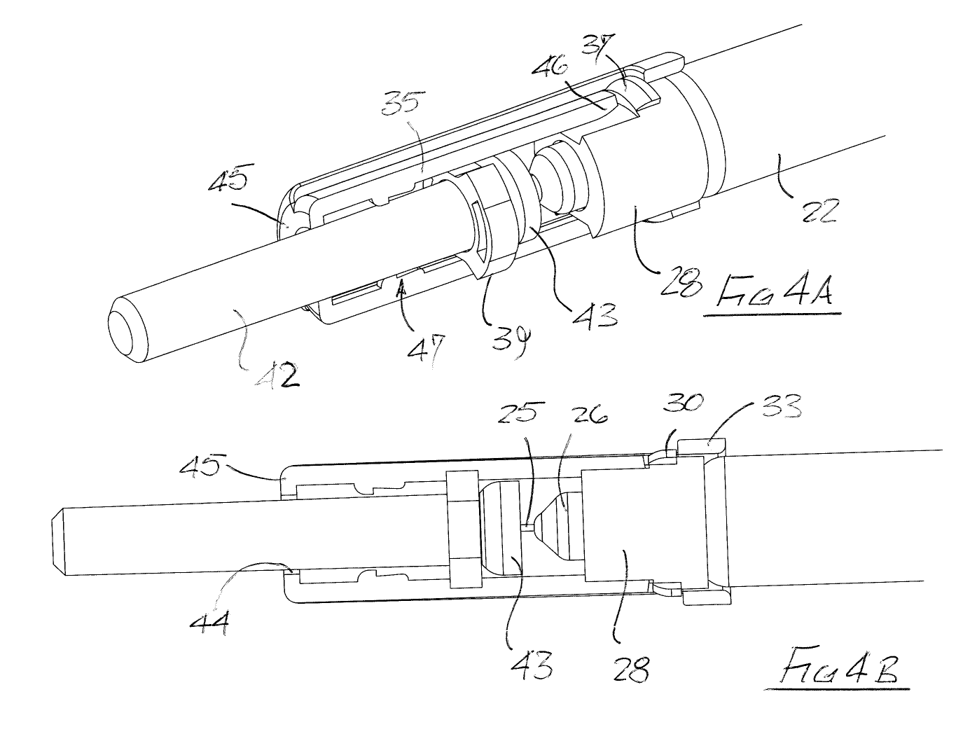

FIGS. 4A and 4B correspond to FIGS. 3A and 3B but in a first stage of preparing the device for performing an injection;

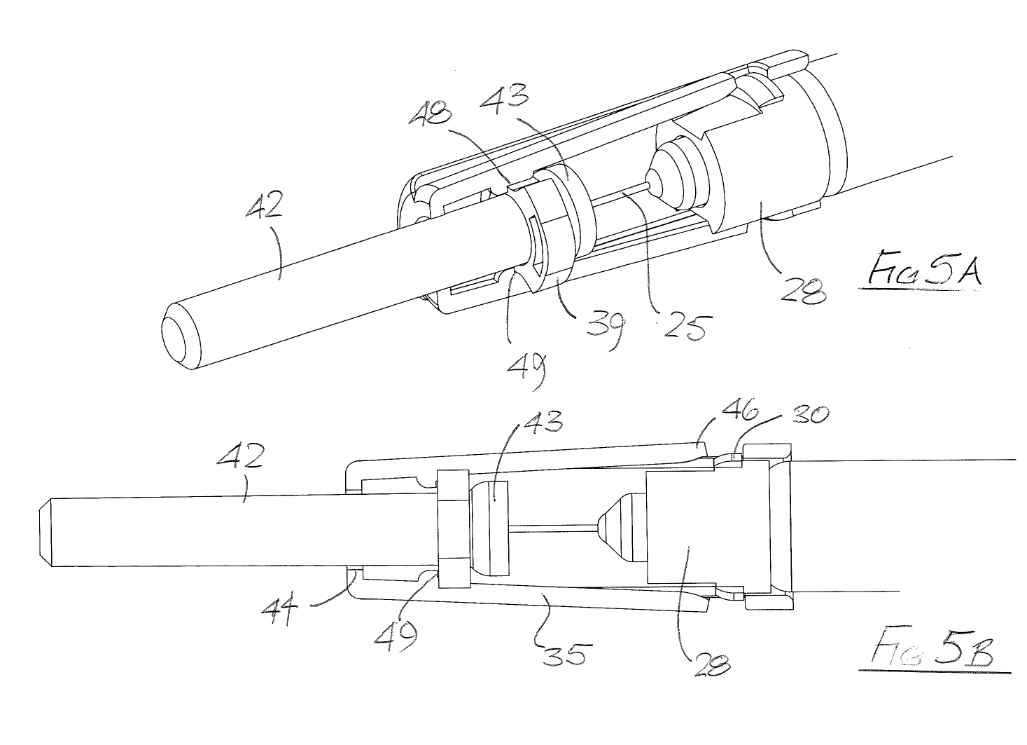

FIGS. 5A and 5B correspond to FIGS. 3A and 3B but with the device in its set condition;

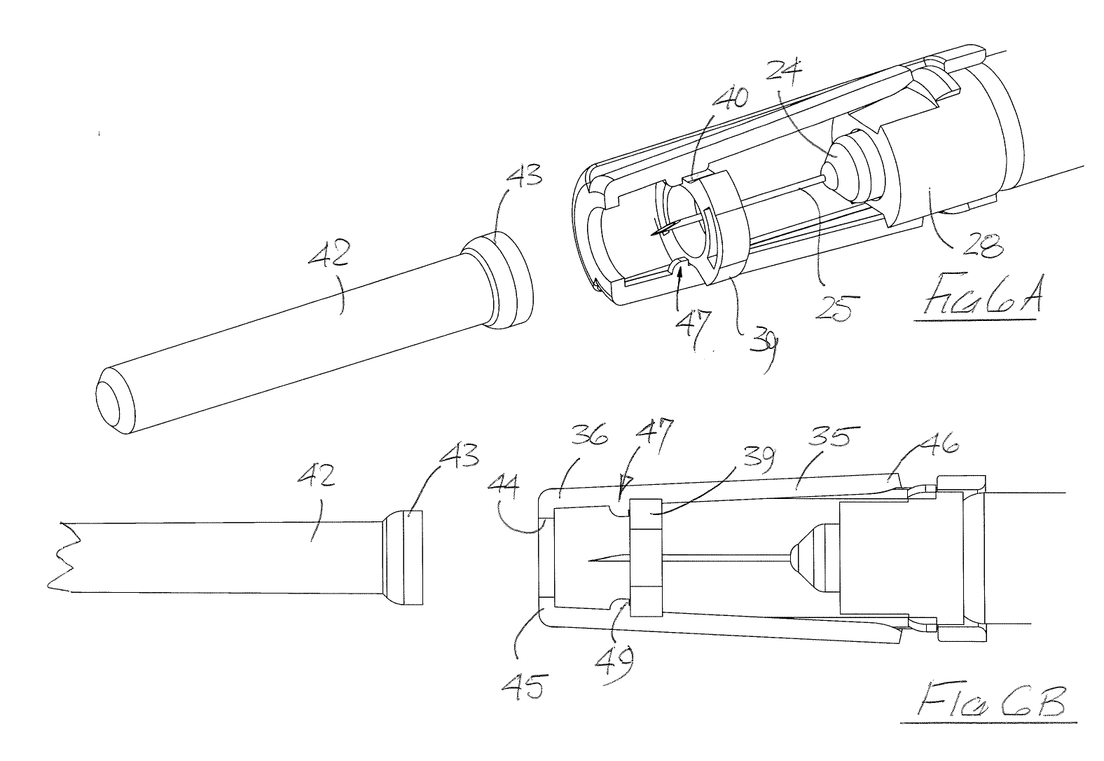

FIGS. 6A and 6B correspond to FIGS. 3A and 3B but having the needle cover removed from the device;

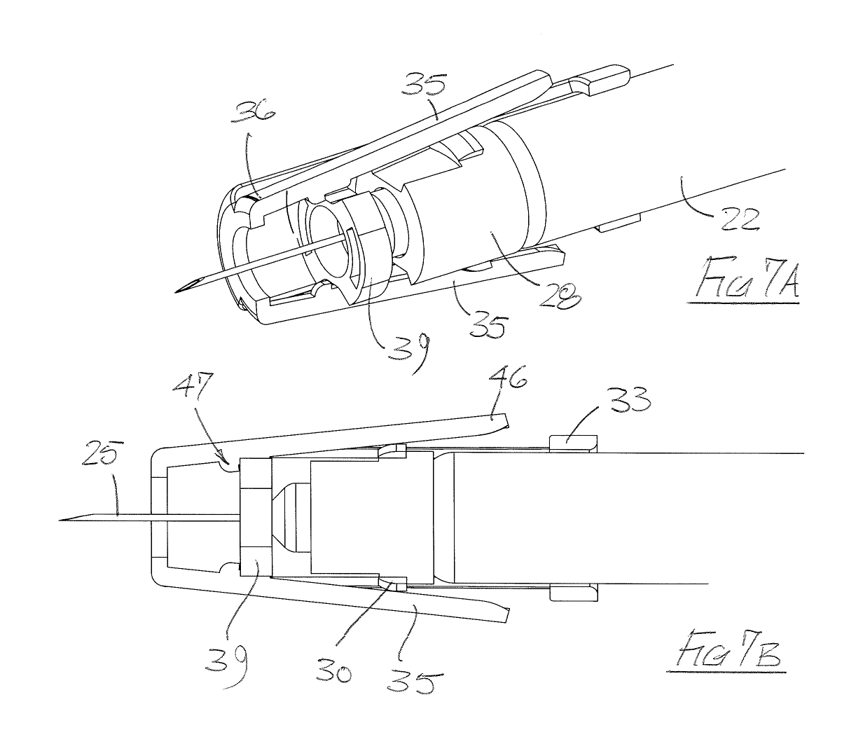

FIGS. 7A and 7B correspond to FIGS. 3A and 3B but showing the components of the device in the course of performing an injection;

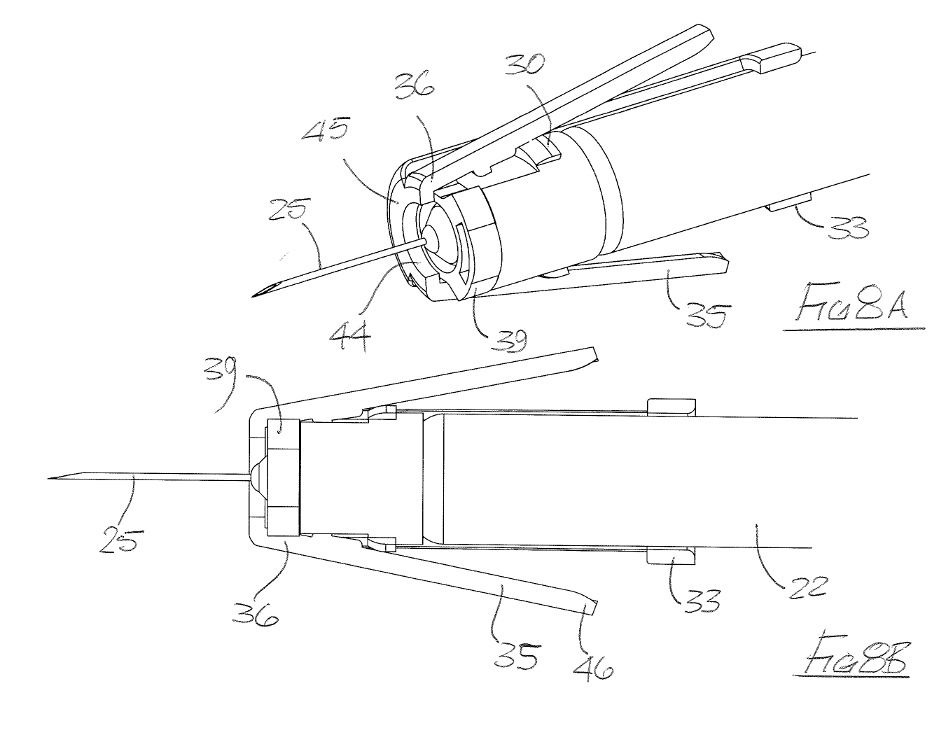

FIGS. 8A and 8B correspond to FIGS. 3A and 3B but with the needle projecting fully from the front of the protective shield;

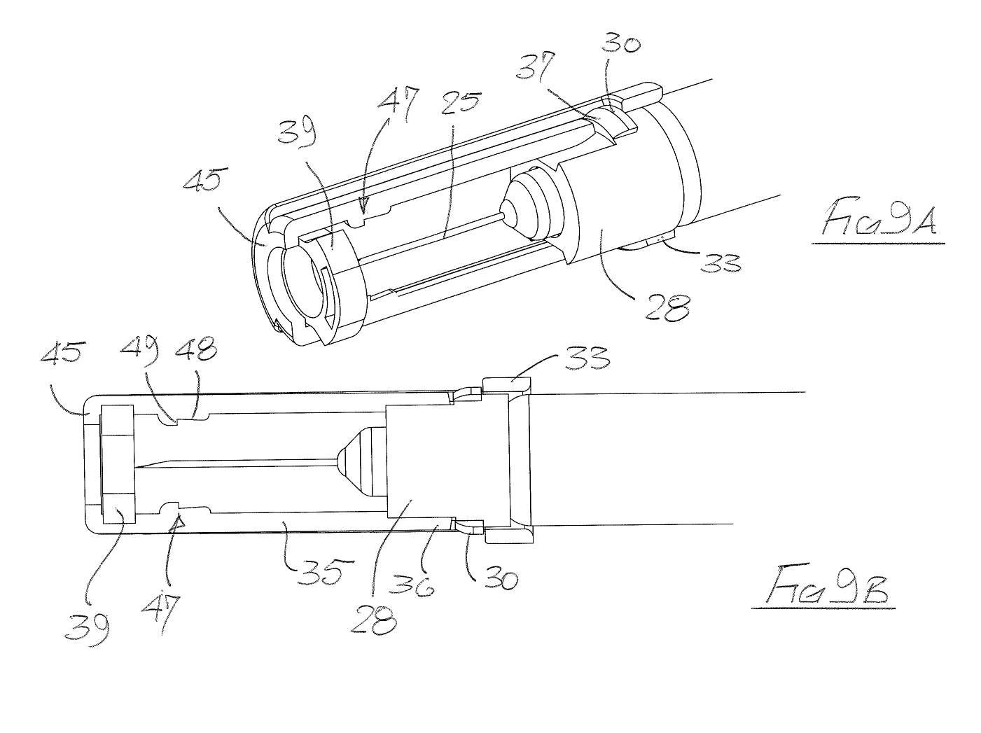

FIGS. 9A and 9B correspond to FIGS. 3A and 3B but showing the components in their relative positions at the completion of an injection;

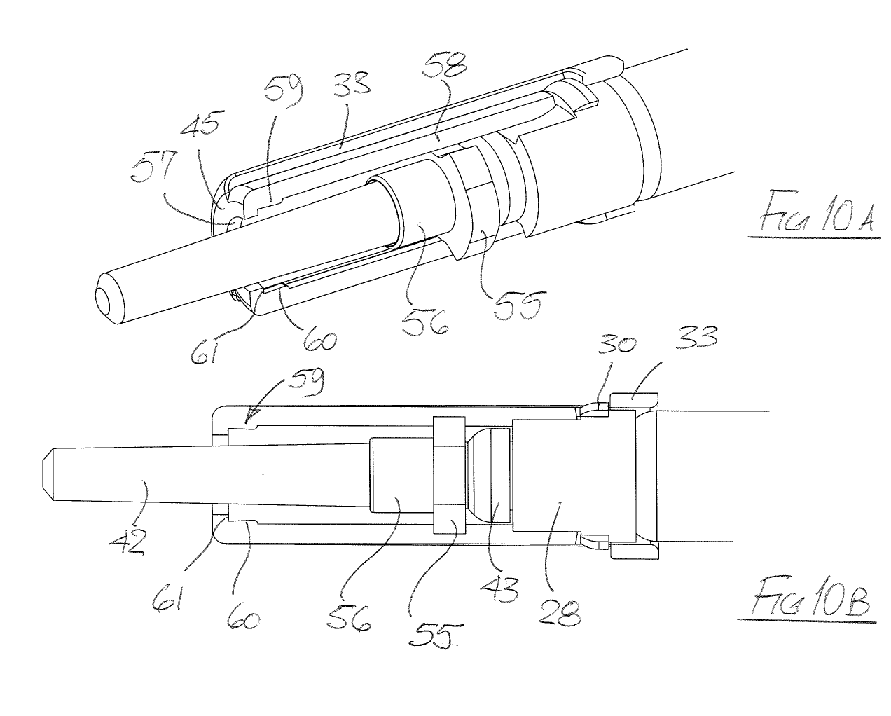

FIGS. 10A and 10B are respectively a cut-away isometric view and an axial section through a second embodiment of safety device, ready for use;

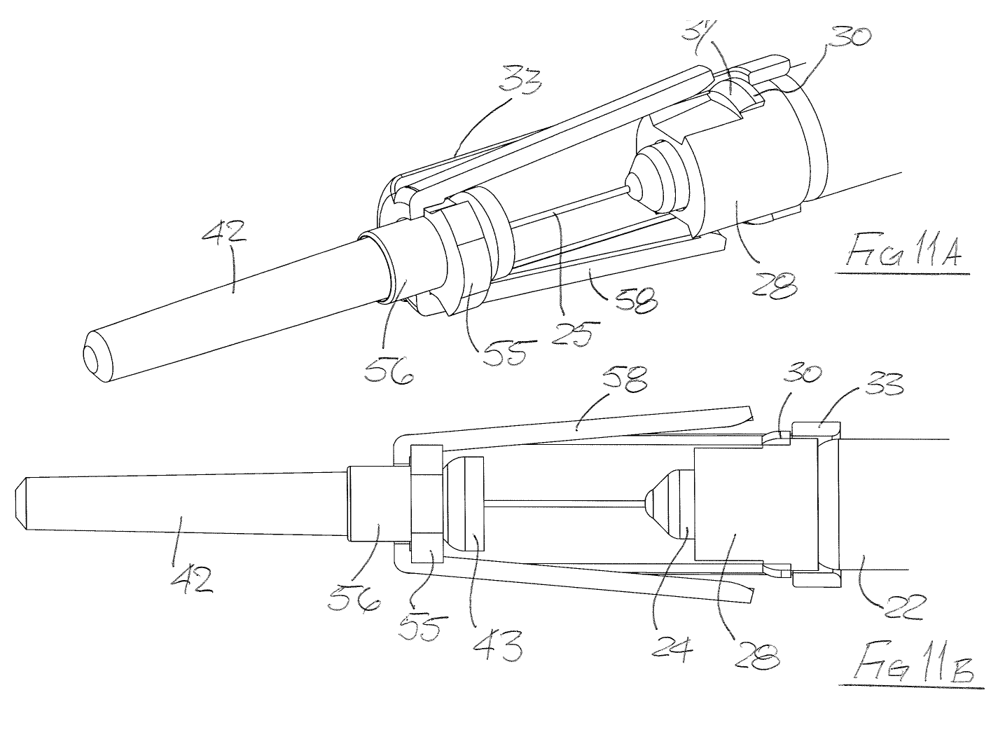

FIGS. 11A and 11B generally correspond to FIGS. 10A and 10B but with the device in its set condition;

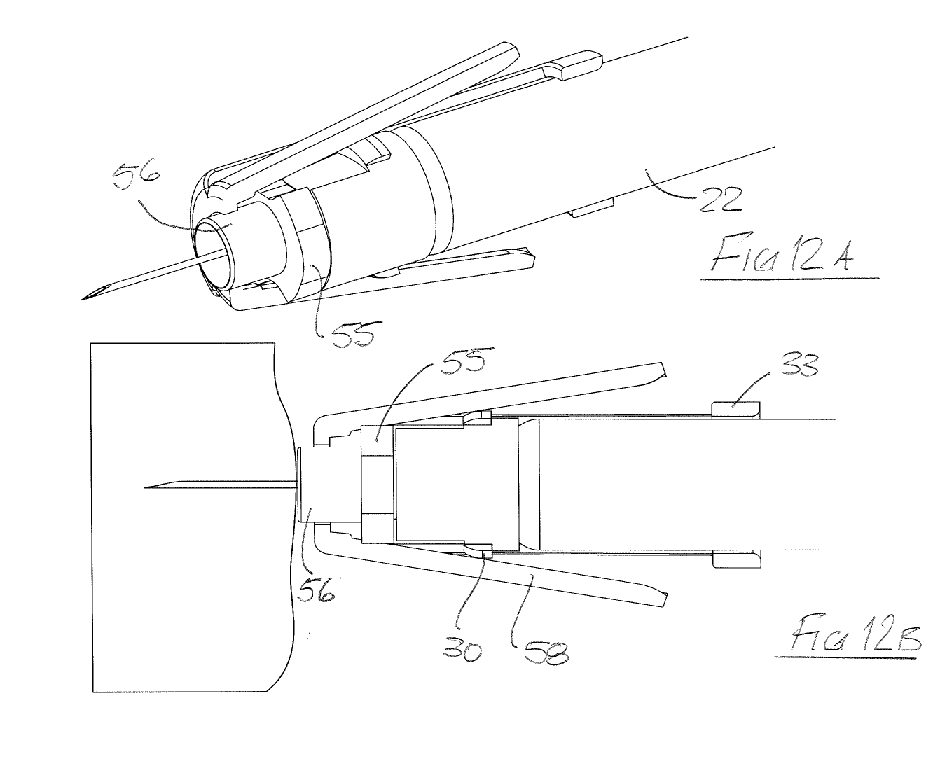

FIGS. 12A and 12B generally correspond to FIGS. 10A and 10B but with the needle projecting fully from the front of the protective shield and in the course of performing an injection;

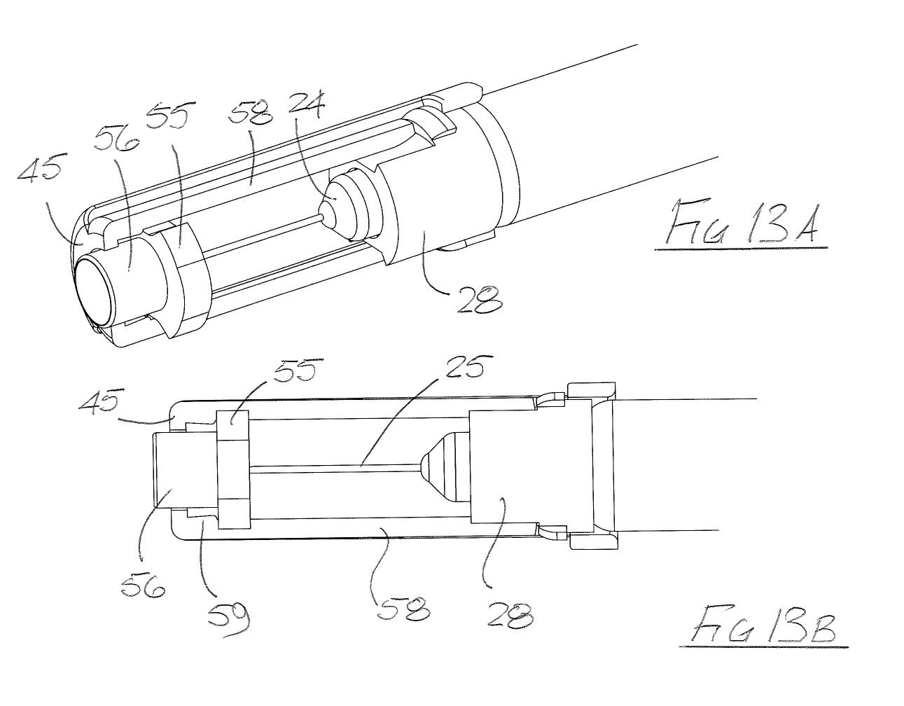

FIGS. 13A and 13B generally correspond to FIGS. 10A and 10B but showing the components in their relative positions at the completion of an injection;

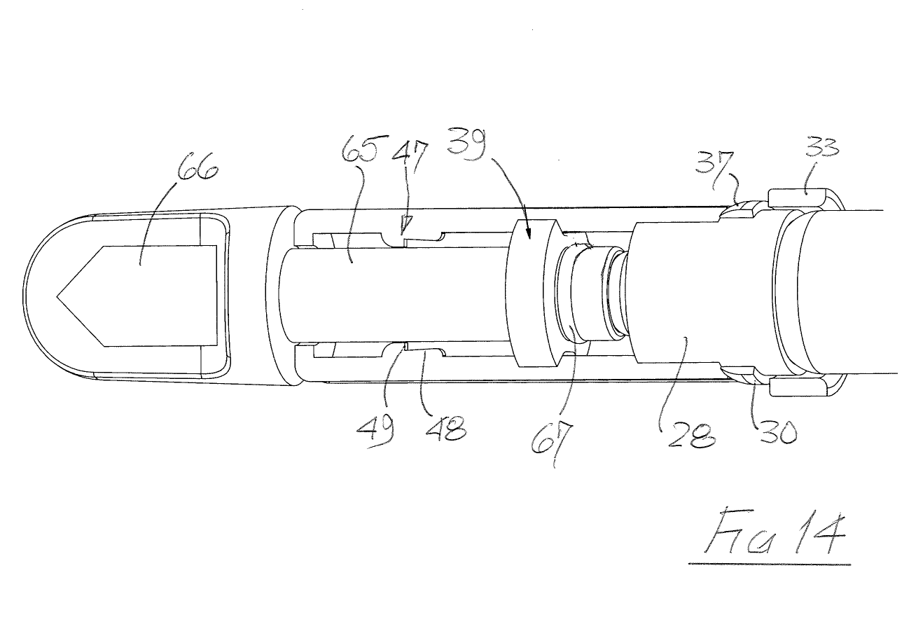

FIG. 14 is a part sectional view of a third embodiment of safety device, with the components in their initial positions; and

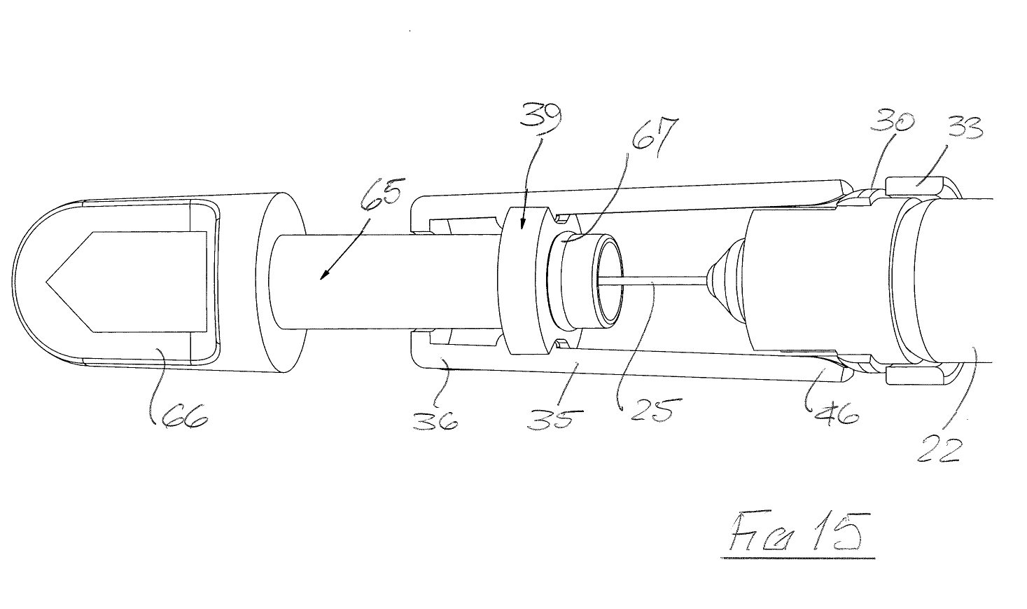

FIG. 15 corresponds to FIG. 14 but with the components in their set position, with the device ready for use in performing an injection.

The first embodiment of safety device of this invention is shown in FIGS. 1 to 9. The safety device 20 is shown mounted on a conventional medical syringe 21 having a barrel 22 and a plunger 23 supporting a piston within the bore of the barrel. At its forward end, the syringe has a nose 24 carrying a staked-in hollow needle 25 through which a liquid drug is discharged on depression of the plunger. The forward end of the nose 24 has an annular rib 26 for a purpose to be described below. Typically, the syringe will be pre-filled during manufacture with a liquid drug and then will be stored with a cover over the needle. The safety device may also be fitted to the syringe during manufacture or may be fitted subsequent thereto, for example immediately before the syringe is to be used, though still with the cover over the needle.

The safety device 20 has a generally cylindrical mount 28 the external diameter of which is substantially the same as that of the syringe barrel 22. A pair of diametrically opposed ramp surfaces 29 are formed in the external surface of the mount 28, the rearward end of each ramp surface providing an upstanding lug 30, the forwardly directed face of each lug defining a shoulder. The mount has a front wall 31 in which is formed a hole 32 such that the mount may be snap-fitted over the annular rib 26 at the forward end of the syringe nose 24, whereby the mount may be securely carried at the forward end of the syringe, as shown in FIGS. 3 to 9.

The safety device also includes a sleeve 33 arranged for axial sliding movement with respect to the mount 28 and so also with respect to the syringe 21 and staked-in needle 25. The initial needle-shielding position is shown in FIGS. 3A and 3B, and the sleeve may slide rearwardly to a non-shielding position shown in FIGS. 8A and 8B, where part of the needle back from its sharp tip is exposed, so that a medical procedure such as an injection may be performed. The sliding movement of the sleeve may occur as a part of that procedure.

The sleeve has a pair of opposed elongate apertures 34 within which are furnished respective fingers 35 connected to the main part of the sleeve at the forward end 36 thereof, for serving as leaf springs. Though two such apertures each having a respective finger are shown, other numbers of apertures and fingers could be employed, ranging from a single aperture and finger up to three or four apertures and fingers and perhaps even five or more. Each finger 35 is resiliently deformable radially outwardly, as will be apparent from the following description of the device.

The opposed lugs 30 formed on the mount 28 locate in the apertures 34 of the sleeve and serve to prevent removal of the sleeve 33 from the mount, once fitted thereon and with the sleeve in its initial position shown in FIGS. 3A and 3B. The lugs 30 also serve to limit rotational movement of the sleeve with respect to the mount 28. The forwardly directed surface 37 of each lug is of conical form, so as to facilitate fitting of the sleeve to the mount and also to provide a sliding surface for the associated finger, when the sleeve slides with respect to the mount.

A generally ring-shaped control member 39 is disposed within the sleeve 33 and in the initial setting of the device (FIGS. 3A and 3B) is disposed close to the forward end of the mount 28. The external periphery of the control member has a pair of diametrically opposed flanks 40 aligned with the fingers. The control member has a sufficiently large bore 41 to accommodate the main part of a soft needle cover 42 typically made of an industry-standard elastomer. The needle cover has a generally conical enlargement 43 at its rearward end to engage and effect a seal with the nose 24 of the syringe, that part of the needle cover being of sufficient flexibility to allow the enlargement to pass through the bore of the control member 39 when the control member is restrained against movement and a sufficient force is applied to the cover. The main part of the cover is projects forwardly out of an opening 44 formed in the front face 45 of the sleeve 33, to allow manual gripping for removal of the cover from the device.

The rearward end 46 of each finger 35 is formed for engagement with the shoulder formed at the front of each lug 30 of the mount 28, when the finger is in an undeformed condition at the initial setting of the device as shown in FIGS. 3A and 3B—i.e. the finger is radially aligned with the shoulder. As shown in the drawings, the free end of each finger is raked slightly and the shoulder is correspondingly angled such that when the rearward end 46 of the finger engages the shoulder, the finger is encouraged to move deeper into engagement. A small amount of free play may be provided between the rearward end of each finger and the shoulder, so permitting a limited amount of free movement of the shield in its shielding position.

Each finger 35 has an internal formation 47 formed partway between the ends of the finger but nearer the forward end thereof. The formation has a surface 48 projecting internally of the sleeve, the formation being rounded at the rearward end of that surface. At the forward end of that surface there is an abutment 49 for engagement by the control member 39, during use of the device as will be described below.

In this embodiment, each of the mount, sleeve and control member is made of a moulded polymeric material. The fingers 35 are resiliently deformable radially outwardly by flexing of those fingers but in the initial position shown in FIGS. 3A and 3B, the fingers are undeformed—in their as-moulded unstressed condition. Thus, the device may be stored in that condition without the fingers suffering from a loss of resilience, which otherwise would occur through storage if the fingers were deformed, unless made of a material which does not lose resilience if stressed for an extended period.

The operation of the safety device 20 described above will now be explained, following the fitting of the device to a syringe 21. The initial setting of the device is shown in FIGS. 3A and 3B, with the sleeve 33 in its forward needle-shielding position so as to confer protection to the needle. In this setting, the fingers 35 extend substantially parallel to the axis of the sleeve and mount 28 and are in an undeformed condition, as mentioned above, axially aligned with the shoulders of the lugs 30. The needle cover 42 is in position, projecting beyond the front face 45 of the sleeve 33 and effecting a seal to the sharp tip of the needle 25. The rear end of the cover also effects a seal to the nose 24 of the syringe. The cover extends through the control member 39, which is disposed adjacent the enlargement 43 of the cover close to the mount 28. The rearward end of the fingers may bear against the shoulders of the mount lugs 30 and block rearward movement of the sleeve, though there may be a small amount of free play for the sleeve as mentioned above. This is the storage position for the device.

When an injection is to be performed, the safety device is prepared by grasping the projecting part of the needle cover 42 and pulling the entire cover out of the device. Initially, the enlargement 43 of the cover draws the control member 39 forwardly within the sleeve 33 (FIGS. 4A and 4B), until the control member reaches the formation 47. Continued pulling on the cover draws flanks 40 of the control member under the surfaces 48 of the fingers, so driving the fingers radially outwardly, as shown in FIGS. 5A and 5B. This lifts the rearward ends 46 of the fingers clear of the shoulders of the lugs 30 of the mount 28. The control member and fingers are now in their set position allowing rearward movement of the sleeve with respect to the syringe.

The control member 39 is restrained from further forward movement by the abutments 49 of the formations 47. Continued pulling on the cover 42 draws the enlargement 43 through the bore 41 of the control member, with the enlargement flexing as needed to achieve this, while the control member is restrained in its set position. The cover when wholly free of the device may be discarded (FIGS. 6A and 6B). The overall device is now ready for performing an injection, but with the needle still protected by the sleeve 33.

An injection is performed by offering the front face 45 of the sleeve 33 to an injection site. The syringe is then pushed forwardly while the sleeve remains stationary bearing against the injection site. There is thus relative movement between the sleeve 33 and the syringe 21, as shown in FIGS. 7A and 7B, the fingers 35 sliding along the lugs 30 during this action and so being resiliently deformed radially outwardly and storing energy within the fingers. Shortly after the relative position shown in FIGS. 7A and 7B, the front wall 31 of the mount 28 contacts the control member 39 and continued forward pushing of the syringe drives the control member 39 within the sleeve until the control member contacts the internal surface of the front face 45 of the sleeve (FIGS. 8A and 8B). The needle 25 is thus projecting to the maximum extent from the sleeve 33 and depression of the plunger 23 performs the injection of the liquid drug at the injection site.

Once the injection has been completed, the syringe is moved away from the injection site, so withdrawing the needle therefrom. Initially, the front face 45 of the sleeve 33 stays in contact with the injection site by virtue of the fingers 35 releasing their stored energy in effect to move the sleeve forwardly with respect to the mount 28 and syringe 21. When the needle leaves the injection site, the sleeve is moved fully forwardly with respect to the syringe by the restorative force exerted by the fingers 35, which return to their undeformed condition as shown in FIGS. 9A and 9B and thus block subsequent rearward movement of the sleeve. During this action, the control member 39 remains at the forward end of the sleeve, in its final position. The final position of the sleeve relative to the syringe and mount is thus exactly the same as the starting position of the sleeve, but the sleeve cannot move rearwardly once more as such movement is blocked by the fingers being axially aligned with the shoulders.

FIGS. 10 to 13 show the second embodiment which is generally similar to that described above and like parts are given like reference characters; those parts will not be described again in relation to this second embodiment.

In this second embodiment, there is a control member 55 which corresponds to control member 39 of the first embodiment but which has a forwardly projecting tube 56. Also, the opening 57 at the front face 45 of the sleeve 33 is of a sufficient diameter to allow that tube 56 to project therethrough, when the control member 55 is in its set position, fully forwardly within the sleeve. The fingers 58 correspond to fingers 35 of the first embodiment but do not have formation 47 partway between the ends thereof; rather, there is a corresponding formation 59 at the forward end of each finger. This formation has a surface 60 and an abutment 61 defined by the front face of the sleeve.

The operation of the device is essentially the same as that described above in relation to the first embodiment. Before use, the components of the device are in the relative positions shown in FIGS. 10A and 10B, with the control member 55 in its storage position. The needle cover 42 is removed from the device when an injection is to be performed by pulling the cover axially forwardly, and this draws the control member forwardly to its set position, where the control member bears against the abutment 61 at the forward end of the sleeve, so displacing the fingers radially outwardly clear of the shoulders of the lugs 30 (FIGS. 11A and 11B). Continued pulling on the cover draws the cover through the bore and tube 56 of the control member, the enlargement of the needle cover deforming as necessary to allow this to happen, until the cover comes free. The device is now in its set position ready for use, with the tube 56 projecting from the front face 45 of the sleeve 33.

An injection is performed by offering the projecting control member tube 56 to an injection site and then pushing the syringe forwardly towards that site. This moves the needle forwardly to penetrate the site, the fingers of the sleeve being displaced radially outwardly to store energy as the mount 28 slides forwardly within the sleeve until the mount engages a stop (not shown) provided within the sleeve. There is then a gap between the forward end of the mount and the internal face at the front of the sleeve into which the control member may move with continued pressure on the syringe, taking the control member to its final position as shown in FIGS. 12A and 12B.

On completion of the injection, the syringe is moved away from the injection site and the stored energy in the fingers returns the sleeve to its initial position as described with reference to the first embodiment. The control member 56 remains near the forward end of the sleeve 33, but immediately rearwardly of the formations 59 at the forward end of the sleeve, as shown in FIGS. 13A and 13B, so allowing the fingers to return to their undeformed condition, axially aligned with the shoulders of the mount, so blocking subsequent rearward movement of the sleeve with respect to the syringe.

Though in the foregoing embodiment the control member 55 is furnished with a sleeve 56, the control member could instead have some other forward projection so long as that projection is able to pass through the opening at the forward end of the sleeve. For example, a plurality of forwardly projecting separate pegs, or even just one such peg, could be provided on the control member.

FIGS. 14 and 15 show a third embodiment which utilises a different kind of needle cover 65. Apart from the needle cover, this third embodiment corresponds to the first embodiment and will not therefore be described again in detail here.

The forward end of the needle cover 65 of this embodiment is formed as a tab 66 to facilitate gripping, for the withdrawal of the cover from the device. The rearward end of the cover has an external annular ring 67 of a slightly greater diameter than the bore 41 of the control member 39, such that the control member will be drawn forwardly with the needle cover on pulling on the tab 66 to release the cover from the device. When the control member 39 arrives at the set position, the control member engages the formations 47 of the fingers 35, so resisting further forward movement of the control member. The cover will then deform slightly to allow the ring 67 to pass through the bore of the control member so leaving the control member in its set position shown in FIG. 15. The rearward end of the cover is adapted for sealing to the nose 24 of the syringe, in much the same way as the enlargement 43 of the first embodiment.

In all other respects, this third embodiment corresponds to the first embodiment and is used in the same way. As an alternative to the ring 67, the cover may have other formations which allow the control member to be drawn forwardly by the needle cover, or it would be possible to rely solely upon friction between the needle cover and the control member, to perform this function.

In the foregoing embodiments of the invention the fingers are formed of a polymeric material which cannot be stored with the fingers deformed from their as-moulded state else the fingers will take a “set” and no longer be able to return to their as-moulded state. It would be possible to provide the sleeve with fingers of a material not displaying this characteristic and so which may be stored with the fingers stressed without losing the ability to return to their initial state. Such a material might be an advanced polymer or perhaps a metal. In this case, it may not be necessary for the control member to be moved to its set position from a storage position to prepare the device for use; the control member may instead be stored in its set position.

1. A safety device for shielding a medical needle having a sharp tip, which device comprises:
a needle mount for directly or indirectly supporting a medical needle;
a needle shielding sleeve for surrounding the needle and arranged coaxially with the mount so that a force applied to the sleeve slides the sleeve from an initial needle shielding position to a non-shielding position whereat at least the tip of the needle is exposed beyond the sleeve;
an abutment surface and a sliding surface provided on one of the sleeve and mount;
a radially deformable resilient finger provided on the other of the sleeve and mount, said finger being configured for radial alignment with the abutment surface or the sliding surface, a part of the finger being in radial alignment with the abutment surface when the finger is not radially deformed, thereby blocking movement of the sleeve from in its initial needle shielding position;
a control member slidably housed within the coaxial arrangement of the sleeve and mount and having a set position with respect thereto at which the control member engages with the finger to cause radial deformation thereof so allowing movement of the sleeve from its initial position, said deformation also causing a part of the finger to move into radial alignment with the sliding surface, and movement of the sleeve towards its non-shielding position causes increased deformation of the finger thereby generating a restorative force between the finger and the sliding surface that urges the sleeve back to its initial needle shielding position, said movement of the sleeve from the initial position causes the control member to be displaced from the set position and thereafter allows the finger to return into radial alignment with the abutment surface on return of the sleeve to its initial position, thereby blocking subsequent movement of the sleeve; and
displacement means for effecting movement of the control member from a starting position before use of the device to the set position.

2. A safety device as claimed in claim 1, wherein the displacement means acts axially on the control member to cause axial movement thereof to its set position.

3. A safety device as claimed in claim 1, wherein the displacement means comprises a tubular member surrounding a supported needle and co-operating with the control means, the tubular member being removable from the device following movement of the control member to its set position.

4. A safety device as claimed in claim 3, wherein the tubular member comprises a removable needle cover which co-operates with a supported needle to confer protection to said needle during storage of the device.

5. A safety device as claimed in claim 4, wherein the needle cover is interconnected with but releasable from the control member.

6. A safety device for shielding a medical needle having a sharp tip, which device comprises:
a needle mount supporting a medical needle or adapted directly or indirectly to support a medical needle;
a needle shielding sleeve for surrounding a supported needle and arranged coaxially with the mount for sliding movement between an initial needle shielding position and a non-shielding position whereat at least the tip of a supported needle is exposed beyond the sleeve;
resilient means for urging the sleeve to its needle shielding position;
a control member for the resilient means, the control member being slidably housed within the coaxial arrangement of sleeve and mount and having a set position axially spaced from a storage position, the resilient means being in a substantially undeformed condition when the control member is in its storage position but being partially deformed when the control member in its set position; and
a needle cover fitted on to the needle to confer protection thereto but removable from the needle prior to use of the device and arranged so that removing the needle cover effects movement of the control member to its set position.

7. A safety device as claimed in claim 6, wherein removing the needle cover from the needle initially draws the control member axially to its set position, whereafter the needle cover is released from the control member.

8. A safety device as claimed in claim 7, wherein the control member is annular and has an opening therethrough, and the needle cover is resiliently deformable and passes through said opening in the control member.

9. A safety device as claimed in claim 8, wherein the rearward end of the needle cover is enlarged but deforms inwardly to allow the rearward end of the cover to pass through the opening.

10. A safety device as claimed in claim 8, wherein the rearward end of the needle cover is frictionally engaged with the control member.

11. A safety device as claimed in claim 8, wherein the rearward end of the needle cover includes an annular rib engageable with the control member and is deformable inwardly to allow the rearward end of the cover to pass through the control member.

12. A safety device as claimed in claim 6, wherein the rearward end of the needle cover effects a seal to the needle mount.

13. A safety device as claimed in claim 6, wherein the forward end of the needle cover projects forwardly from the sleeve to allow the manual gripping thereof.

14. A safety device as claimed in claim 6, wherein the needle mount pushes the control member from its set position in the course of the sleeve moving rearwardly with respect to the needle mount.

15. A safety device as claimed in claim 6, wherein the control member includes a projection extending forwardly from the control member and when the control member is in its set position the projection projects from the forward end of the sleeve.

16. A safety device as claimed in claim 15, wherein the projection moves the control member rearwardly away from its set position in the course of the rearward movement of the sleeve.
